(19) United States
(12) Patent Application Publication
(10) Pub. No.: US 2014/0154132 A1
(43) Pub. Date: Jun. 5, 2014

(54) Sterilization System and Components for Express Cycle Sterilization

(71) Applicant: CASE MEDICAL, INC., South Hackensack, NJ (US)

(72) Inventors: Marcia A. Frieze, Alpine, NJ (US); Allan S. Frieze, Alpine, NJ (US)

(73) Assignee: Case medical, Inc., South Hackensack, NJ (US)

(21) Appl. No.: 13/778,362

(22) Filed: Feb. 27, 2013

Related U.S. Application Data
(60) Provisional application No. 61/732,533, filed on Dec. 3, 2012.

(51) Int. Cl. B65D 6/00; A61L 2/20; B65D 21/02

(52) U.S. Cl. CPC B65D 7/00; A61L 2/208; B65D 21/0233
USPC 422/28; 220/23.87; 220/23.9; 220/677

(57) ABSTRACT

A sterilization container, insert therefor, and systems are described, wherein at least one of the container or the insert therefor at the contact point between them is constructed of a conductive, non-corrosive material, most preferably stainless steel, while the bulk of the container or insert therefore is constructed of a non-conductive material, most preferably anodized aluminum. The container, insert, and whole systems are suitable for use in gas plasma sterilization procedures, but especially in those procedures that include a hydrogen peroxide gas plasma “express” cycle.

### FIELD OF THE INVENTION

The present invention relates to the field of sterilization containers and inserts therefore, particularly in the area of hydrogen peroxide gas plasma sterilization.

### BACKGROUND OF THE INVENTION

Sterilization containers have been a big challenge for hydrogen peroxide and hydrogen peroxide gas plasma sterilization and have been particularly a greater challenge for “express” cycles due to the short cycle parameters and the reduced concentration of hydrogen peroxide. Prior sterilization containers and sterilization container systems are disclosed in patents and patent applications owned by Case Medical Inc, such as for example U.S. Pat. No. 5,968,459; U.S. Pat. No. 6,468,482; U.S. Pat. No. 6,589,477; US 2002/0136679; US 2005/0238530; and WO 2001062302; all of which are incorporated herein in their entirety except to the extent as contradicted by the express disclosure herein.

Sterilization in the prior containers and systems was suitable for steam, gas, and hydrogen peroxide gas plasma as discussed in these prior patents. increased circulation and sterilant penetration was necessary to achieve sterility within a sealed sterilization container when hydrogen peroxide gas plasma systems were utilized. However, the increased circulation and sterilant penetration did not lead to the desired outcome in the “express” cycle. It was found that when mated surfaces of anodized aluminum inoculated with spores at the contact points, sterilization did not result in full lethality when using express cycles. It has now been determined that stainless steel and other conductive metals, whether passivated for corrosive resistance or not, achieve the desired outcome of microbial kill of mated inoculated surfaces. Anodized aluminum was the preferred metal for both container and inserts in the previous patents for the following reasons: anodized aluminum itself is highly thermo-conductive and provides a passive layer that protects the device(s) to be sterilized from corrosion, discoloration, and enhances the useful life of the aluminum sterilization container device. Anodized aluminum also prevents galvanic reactions when dissimilar metals are in contact therewith, but the anodized aluminum non-conductive. It was our speculation that if one of the mated surfaces was conductive the plasma effect would be enhanced and allow for sterilization at the lower temperatures, lower concentrations of hydrogen peroxide, and/or shorter cycle times demanded in the more recent “express” cycles and allow for continued use of non-conductive materials, especially anodized aluminum. More recently, we achieved feasibility when one of the mated surfaces was stainless steel and the other of the mated surfaces was anodized aluminum.

There is a counter indication to utilize mated anodized aluminum surfaces with 0 tolerance in “express” hydrogen peroxide gas plasma cycle due to the shortness of the exposure time of the cycle. While our previous container had success in other STERRAD sterilizers and other STERRAD gas plasma cycles, including the STERRAD 100 NX flex and standard cycles, it did not pass in the STERRAD 100 NX Express cycle. The STERRAD 100S sterilizer, which has a cycle time of 55 minutes, and the STERRAD NX sterilizer, which has a 28 minute cycle time, utilize a 59% hydrogen peroxide concentration that internally concentrates the sterilant to 90%. The STERRAD 100 NX sterilizer, which also utilizes a 59% hydrogen peroxide, has a standard cycle which has a cycle time of 47 minutes, a flex cycle which is 42 minutes and the Express cycle which has a 24 minute cycle time, which translates into a 3 minute exposure time to the sterilant in the express cycle. What we found is that when the mated anodized aluminum surfaces were inoculated with spores (at the contact points), they did not achieve sterility in the “express” cycle, although they did work in the non-express sterilization cycles, The “zero” tolerance of the mated anodized surfaces did not allow for the potential residual microorganisms at the container/basket interface contact points, even though the instruments in the basket were sterile. Anodized aluminum containers with anodized aluminum inserts with mated surfaces, have been shown to have an unsuccessful outcome in hydrogen peroxide sterilization abbreviated, “express” cycles.

While anodized aluminum is an effective material for steam and gas sterilization as well as hydrogen peroxide in most circumstances, it was found in various studies to be a deterrent when mated anodized aluminum surfaces were challenged h biological indicators with spores in hydrogen peroxide, low temperature, “express” cycles with a short exposure time and reduced concentration of hydrogen peroxide sterilant. Non-anodized aluminum, non-treated metal, is effective to achieve sterility of inoculated mated surfaces. However when aluminum is not treated, i.e., anodized, it is prone to corrosion and the formation of aluminum oxide which forms a powdery residue on surfaces. This residue could be a detriment to patients undergoing a surgical procedure. Thus, an alternate sterilization container and/or insert and/or component and/or complete system is needed in order to he suitable for use in an “Express” sterilization procedure.

### DETAILED DESCRIPTION OF THE INVENTION

It was our determination, that with hydrogen peroxide gas plasma, conductivity at the contact points between surfaces with zero tolerance may be necessary in these “express” cycles to enhance the plasma effect. As used herein, “conductivity” of a metal means that the metal permits the lethal effect of the sterilant to reach contaminants at the contact points between itself and another surface, so that a contact point between metal surface that is conductive and one that is not, an adequate cidal effect is seen at the contact point, whereas the cidal effect at a contact point between two non-conductive materials may not be adequate at the contact point in an express cycle. Anodized aluminum which is treated for corrosion resistance is non-corrosive but also non-conductive, while non-anodized aluminum is an untreated metal that is conductive, but it is also prone to corrosion. While non-anodized aluminum surfaces are conductive, such surfaces are a challenge to clean and decontaminate using soap and water following hospital protocols. All medical devices must be thoroughly cleaned and rinsed before sterilization may occur to remove residual soil and bioburden. As untreated metal is prone to corrosion and the formation of aluminum oxide powdery residue, it presents potential detriments to surgical patients because of galvanic reactions and possible formation of granulomas from the residue in invasive surgery. Recognizing the importance of health and safety issues, the use of non-anodized aluminum for the manufacture of sterilization containers and/or inserts for reusable packaging of surgical devices was not an option for use in express cycle sterilization.

Stainless steel has proven to be safe and effective for surgical devices. Stainless steel has also proven to be a plasma field effect conductive metal. Various materials were tested and considered as an option to aluminum such as stainless steel, Delrin, silicone, and plastic at the mated surface point (contact point). While the anodized aluminum with extended circulation per the inventor's previous pattern is a factor as well, the addition of a conductive stainless steel material contributed to obtaining a successful outcome in the “express” cycle when mated surfaces were challenged. In contrast, the other non-conductive materials tested, did not meet the required outcome. In fact, even though mated anodized aluminum surfaces were considered to be a deterrent to express cycles, stainless steel and anodized aluminum inserts of the same design and configuration when mated together were proven to be more effective in replicate studies when the mated surfaces were challenged.

While other materials can be utilized for the same purpose as the stainless steel (conductivity of the cidal effect of the plasma field), such as silver, gold, titanium, copper, platinum as well as brass, nickel, bronze, iron and non-anodized aluminum, such materials have limitations because of either cost or corrosion, making them unsuitable for general use in sterilization containers, and/or inserts, and/or components. Of course, if cost is not a concern, such alternatives as use of the non-corrosive metals above as well as other alternatives that will be apparent to those of ordinary skill may be used in place of the stainless steel.

The basic issue that the present invention addresses is that two surfaces of non-conductive materials should not be in contact with each other within the confines of the sealed container system when “express” cycles are utilized in hydrogen peroxide gas plasma sterilization, but that, surprisingly, mated surfaces between a conductive and a non-conductive material work very well. The exterior of the container, which comes in contact with the outer environment and is considered non-sterile, does not need to adhere to these restrictions. The invention restrictions pertain to the contact points between (a) the inner contents of the container and (b) the inner surface of the container and/or (c) contact points between multiple items within the container.

Anodized aluminum remains the material of choice for rigid, reusable, sterilization containers for the following reasons: thermal conductivity, light weight, corrosion resistance, relatively low cost, non-porous surfaces, among others. Thus, the existing containers and inserts and components of the aforementioned patents can be now utilized in express cycles by virtue of maintaining the necessary contact points (such as tray insert, foot, handle, the tray surface upon which item to be sterilized that have non-conductive surfaced in contact with the tray, etc.) in accordance with the instant invention disclosure.

As will readily be appreciated by those of ordinary skill, this can be accomplished in a multitude of ways, including, but not limited to:
a) constructing all components of the inner basket or tray system of a conductive metal, preferably stainless steel;
b) constructing the tray (or insert on which instruments to be sterilized are placed) of a conductive metal, preferably stainless steel;
c) constructing the surface or the base of the tray on which instruments to be sterilized are placed and the contact points thereof with the outer container of a conductive metal, preferably stainless steel;
d) constructing the handles and feet which come in contact with the anodized base of the container of a conductive metal, preferably stainless steel;
e) using fasteners and spacers for joining two non-conductive surfaces, which fasteners and joiners are of a conductive metal, preferably stainless steel;
utilizing “feet” for the tray placed inside the container, which feet are made of a conductive metal, preferably stainless steel; among others.

As used herein, an “express cycle” is one which has a significantly shorter cycle time as compared to a full cycle. As differing sterilizers have differing “full cycles” the cycle time for an “express cycle” can vary from system to system. In general, express cycle times have ranges having a minimum selected from about 2 minutes, about 3 minutes about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, and about 26 minutes and an upper limit selected from about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, and about 30 minutes, provided of course that the selection from the minimum list is the same as or less than that selected from the maximum list. Independently, an “express cycle” in a hydrogen peroxide gas plasma sterilization as used herein utilizes a hydrogen peroxide concentration of at least about 40% up to a about 89%, preferably at least about 59%.

It should be noted that a substantial advantage of the present invention is that containers/inserts/components of the instant invention can be used in express cycle and non-express cycle sterilization using hydrogen peroxide gas plasma as well as in other sterilization systems with other sterilants, meaning that facilities baying needs of multiple types of sterilization units can utilize a single set of containers/inserts/components thereby saving substantially on costs, storage space, as well as avoiding errors in using inappropriate mix and match combinations.

In an exemplary embodiment, a sterilization container system optionally with one or more inserts therefor suitable for use in a hydrogen peroxide plasma sterilization express cycle comprises a sterilization container; and optionally, one or more inserts for placing in said container; said container having four walls, a bottom and a cover, defining an interior of said container; wherein each contact point between said container wall, bottom, and cover which is between two discrete pieces thereby resulting in first mated surfaces is constructed so that such mated surfaces at the contact points thereof has one portion of the mated surface of a conductive metal and the other surface that is mated therewith of a non-conductive metal; and wherein each contact point between any of the container interior walls, bottom, or cover on the one band and any insert therefor placed inside said container on the other hand is so configured that such contact points result in second mated surfaces wherein one of the second mated surfaces is of a conductive metal and the other surface that is mated therewith in said second mated surfaces is a non-conductive metal; and wherein any connectors that have a portion in the interior of said container that result in a third mated surface interior to said container are so constructed as to have one of the third mated surfaces to be of a conductive metal and the other of the third mated surfaces that is mated therewith to be of a non-conductive metal; such that each metal to metal mated surface in the interior of said container is a mating of conductive metal and non-conductive metal. In preferred (non-limiting) embodiments of this type, the conductive metal in each situation is stainless steel and the non-conductive metal in each situation is anodized aluminum. Furthermore, in another non-limiting embodiment, the container walls, top, and bottom are anodized aluminum, said top is substantially anodized aluminum; and further comprising a cover—wall-contact liner which is stainless steel.

I/We claim:

1. A sterilization container for use in a hydrogen peroxide gas plasma sterilization system and compatible with use in an “express” cycle of said sterilization system wherein said container is constructed such that any metal-to-metal mated surfaces at the interior of said container have a first surface that is a conductive metal and a second surface that is mated therewith that is a non-conductive metal.

2. The sterilization container of claim 1 wherein contact points for a tray or insert thereof comprise (a) a conductive metal when the container interior surface is otherwise generally a non-conductive metal or (b) a non-conductive metal when the container interior surface is otherwise generally a conductive metal.

3. The sterilization container of claim 2 wherein portions thereof that are not at the contact point are at least in part made of a non-conductive material.

4. An insert for a sterilization container for use in a hydrogen peroxide gas plasma sterilization system and compatible with an “express” cycle thereof wherein said insert is constructed such that any metal-to-metal mated surfaces thereof have a first surface that is a conductive metal and a second surface that is mated therewith that is a non-conductive metal.

5. The insert of claim 4 wherein contact points thereof for contact with said sterilization container interior comprise (a) a conductive metal when the contact point of the sterilization container comprises a non-conductive metal or (b) a non-conductive metal when the contact point of the sterilization container comprises a conductive metal.

6. The container of claim 1 wherein said conducting metal is stainless steel.

7. The container of claim 1 wherein the non-conductive metal is anodized aluminum.

8. The insert of claim 4 wherein said conducting metal is stainless steel.

9. A method of sterilization in a hydrogen peroxide gas plasma sterilization procedure comprising using the sterilization container of claim 1.

10. A method of sterilization in a hydrogen peroxide gas plasma sterilization procedure comprising using the insert of claim 4.

11. The method of claim 9 further comprising an insert for said container compatible with an “express” cycle thereof wherein said insert is constructed such that any metal-to-metal mated surfaces thereof have a first surface that is a conductive metal and a second surface that is mated therewith that is a non-conductive metal.

12. The method of claim 9 wherein said gas plasma sterilization procedure comprises an “Express” cycle procedure.

13. The method of claim 10 wherein said gas plasma sterilization procedure comprises an “Express” cycle procedure.

14. The method of claim 9 wherein said “express” cycle is a cycle of at least about 2 minutes and not more than about 6 minutes.

15. The method of claim 10 wherein said “express” cycle is a cycle of at least about 2 minutes and not more than about 6 minutes.

16. The method of claim 9 wherein said “express” cycle wherein the concentration of hydrogen peroxide used is at least about 40% to not more than about 89%.

17. The method of claim 10 wherein said “express” cycle wherein the concentration hydrogen peroxide used is at least about 40% to not more than about 89%.

18. A sterilization container system optionally with one or more inserts therefor suitable for use in a hydrogen peroxide plasma sterilization express cycle comprising:
a) a sterilization container; and
b) optionally, one or more inserts for placing in said container;
said container having four walls, a bottom and a cover, defining an interior of said container;
wherein each contact point between said container wall, bottom, and cover which is between two discrete pieces thereby resulting in first mated surfaces is constructed so that such mated surfaces at the contact points thereof has one portion of the mated surface of a conductive metal and the other surface that is mated therewith of a non-conductive metal; and
wherein each contact point between any of the container interior walls, bottom, or cover on the one hand and any insert therefor placed inside said container is so configured that such contact points result in second mated surfaces wherein one of second mated surfaces is of a conductive metal and the other surface that is mated therewith in said second mated surfaces is a conductive metal; and
wherein any connectors that have a portion in the interior of said container that result in a third mated surface are so constructed as to have one of the third mated surfaces to be of a conductive material and the other of the third mated surface that is mated therewith to be of a non-conductive metal;
such that each metal to metal mated surface of the interior of said container is a mating of conductive metal and non-conductive metal.

19. The container system of claim 18 wherein said conductive metal is stainless steel and said non-conductive metal is anodized aluminum.

20. The container system of claim 18 wherein said container walls, top, and bottom are anodized aluminum, said top is substantially anodized aluminum; and further comprising a cover—wall-contact liner which is stainless steel.

21. The container system of claim 20 wherein said inserts are substantially anodized aluminum with stainless steel feet or stainless steel connectors for contacting said insert with the internal surfaces of the bottom or walls of said container.

22. The container system of claim 18 wherein the conductive metal is selected from the group consisting of silver, gold, titanium, copper, platinum, brass, nickel, bronze, iron, and non-anodized aluminum.
